(19) United States
(12) Patent Application Publication
(10) Pub. No.: US 2018/0273490 A1
(43) Pub. Date: Sep. 27, 2018

(54) Process for the Preparation of Eltrombopag Olamine

(71) Applicant: Amneal Pharmaceuticals Company GmbH, Steinhausen (CH)

(72) Inventors: Virendra Kumar Agarwal, Ahmedabad (IN); Lalit Keshav Katariya, Ahmedabad (IN); Gaurav Bhogilal Patel, Ahmedabad (IN); Hitesh Sureshbhai Patel, Ahmedabad (IN)

(21) Appl. No.: 15/774,854

(22) PCT Filed: Nov. 8, 2016
(86) PCT No.: PCT/EP2016/076992

§ 371 Date: May 9, 2018

(30) Foreign Application Priority Data
Nov. 10, 2015 (IN) 4299/MUM/2015

(51) Int. Cl. C07D 231/46; C07C 215/08

(52) U.S. Cl. CPC C07D 231/46; C07B 2200/13; C07C 215/08

(57) ABSTRACT

Salts of eltrombopag are provided. Eltrombopag olamine is prepared from salts of eltrombopag, which are used as intermediates.

### FIELD OF INVENTION

The present invention relates to novel salts of eltrombopag, which is useful as an intermediate for preparing eltrombopag olamine. This invention also relates to a process for preparation of eltrombopag olamine from other eltrombopag salts.

### BACKGROUND OF INVENTION

Eltrombopag (I) is thrombopoietin (TPO) receptor agonist. It interacts with the transmembrane domain of the TPO receptor (also known as cMpl) leading to increased platelet production. Eltrombopag (I) is indicated for the treatment of thrombocytopenia in patients with chronic ITP, thrombocytopenia in patients with hepatitis C infection and in severe aplastic anemia.

Eltrombopag (I) is known by chemical name 3′-{(2Z)-2-[1-(3,4-dimethylphenyl)-3-methyl-5-oxo-1,5-dihydro-4H-pyrazol-4-ylidene]hydrazino}-2′-hydroxy-3-biphenylcarboxylic acid. It is marketed as its olamine salt i.e. bisethanolamine salt, which is formed with two molecules of 2-aminoethanol for one molecule of eltrombopag. Eltrombopag olamine (II) is marketed in USA by Novartis Pharms under trade name Promacta® in the form of oral tablet of 12.5 mg, 25 mg, 50 mg, 75 mg and 100 mg acid. Eltrombopag olamine (II) is represented by following structure.

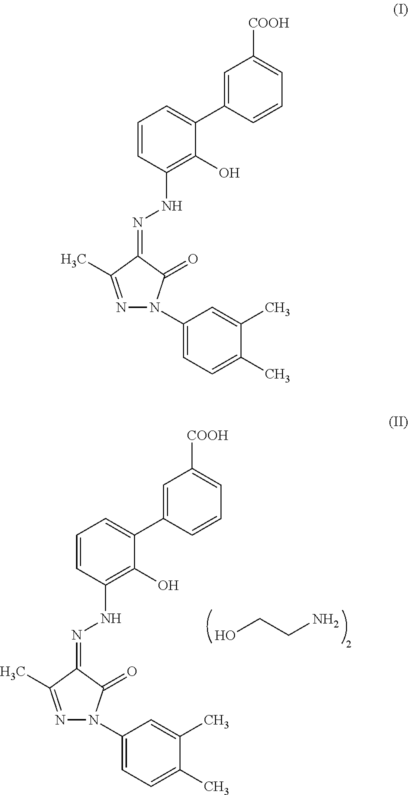

Eltrombopag (I) is disclosed first in U.S. Pat. No. 7,160,870B2. This patent discloses process for preparation of eltrombopag hydrate. However, salts of eltrombopag are not disclosed in the patent.

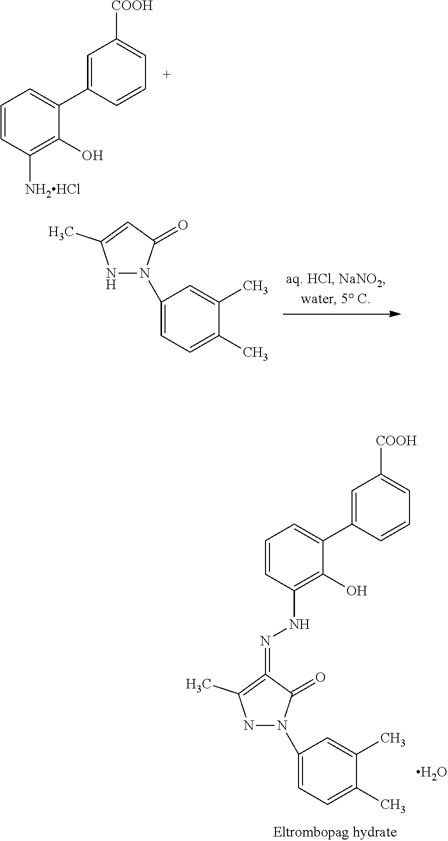

U.S. Pat. No. 7,547,719B2 disclose mono and bis-ethanolamine salt of eltrombopag and its process for preparation.

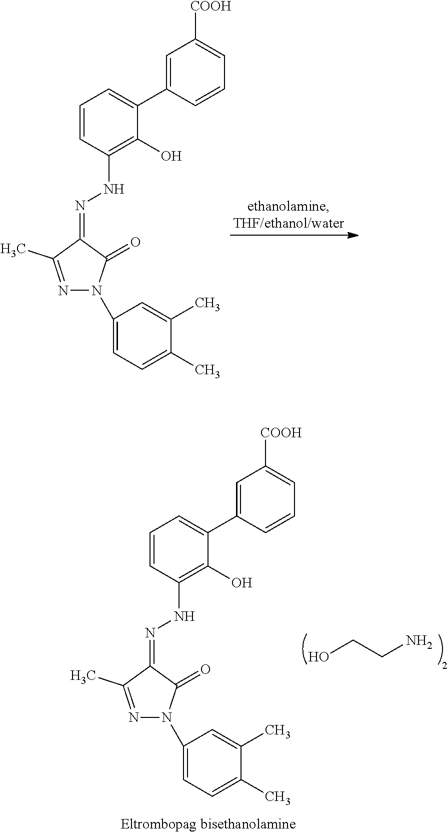

WO2013072921A2 discloses ammonium salt of Eltrombopag and its preparation. IP.com journal, volume 9, issue 12A, 2009 discloses t-butylamine salt and meglumine salt of eltrombopag.

For a pharmaceutical drug process, purity and yield are important factors. There is need in art to provide an economical and cost effective process for preparation of eltrombopag olamine (II), which provides eltrombopag olamine (II) having pharmaceutical grade purity. The present invention provides a process for preparation of pure eltrombopag olamine (II) via formation of novel salts.

### SUMMARY OF THE INVENTION

The present invention provides novel salts of eltrombopag (I).

The present invention provides preparation of novel salt of Eltrombopag.

The present invention provides a crystalline form of eltrombopag triethylamine salt.

The present invention provides use of novel salts of eltrombopag in preparation of eltrombopag olamine (II) which is pure and substantially free of one or more of its corresponding impurities.

The present invention provides a process for preparation of eltrombopag olamine (II) which involves novel salt.

In one aspect, the present invention provides a process for preparation of eltrombopag olamine (II),

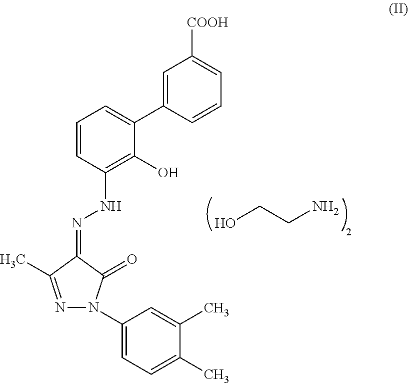

which comprises,
a) diazotizing 3′-Amino-2′-hydroxy-biphenyl-3-carboxylic acid (III) and then condensation with 2-(3,4-Dimethyl-phenyl)-5-methyl-1,2-dihydro-pyrazol-3-one (IV) to give eltrombopag (I),

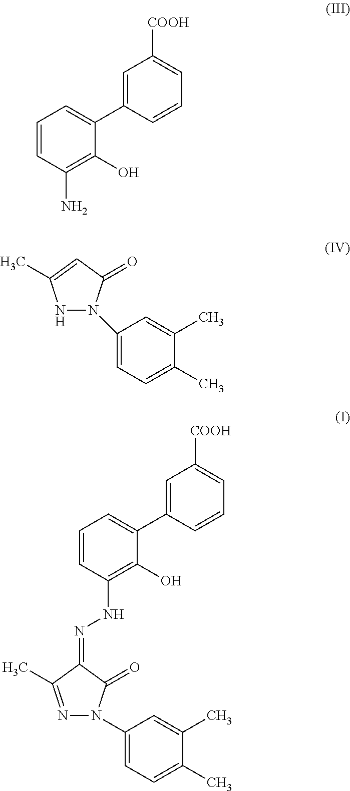

b) converting eltrombopag to eltrombopag salt (V); and

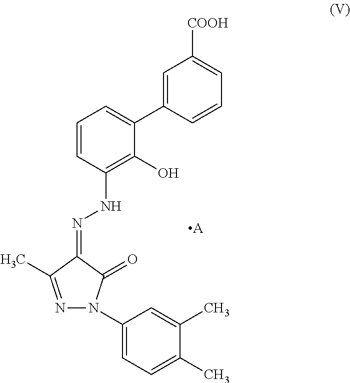

wherein A is base molecule;
c) converting eltrombopag salt (V) to eltrombopag olamine (II).

In another aspect, the present invention provides novel salts of eltrombopag having following structural formula:

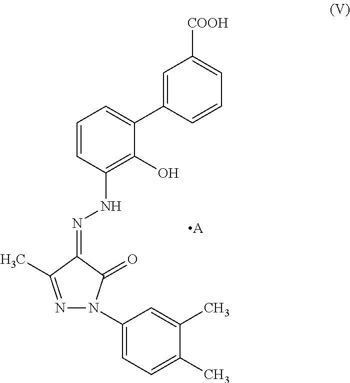

wherein A is base molecule preferably N-alkyl amine.

Particularly following salts are covered in the present invention.

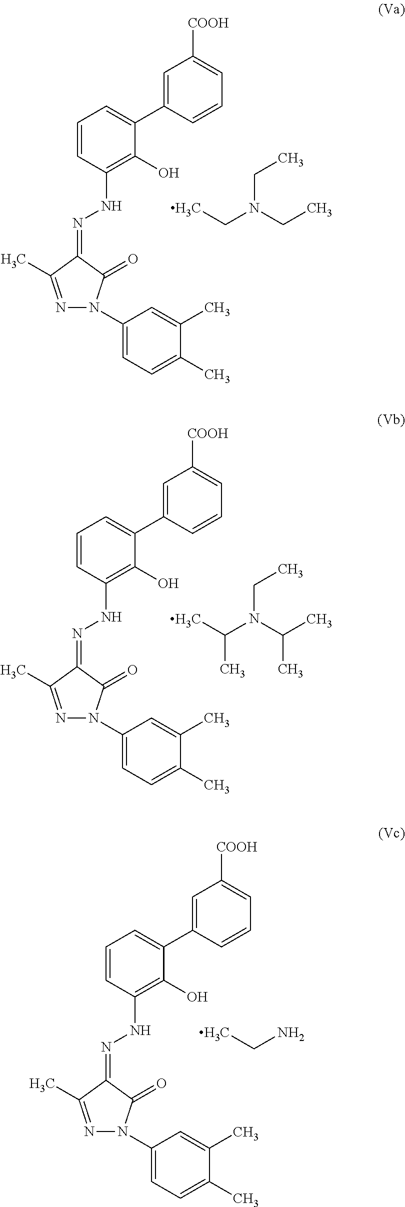

In yet another aspect, the present invention provides use of novel salts of eltrombopag in preparation of eltrombopag olamine (II).

### BRIEF DESCRIPTION OF THE FIGURES

FIG. 1 shows a powder XRD pattern of crystalline eltrombopag triethyamine salt.

FIG. 2 shows a powder XRD pattern of crystalline eltrombopag diisopropylamine salt.

FIG. 3 shows a powder XRD pattern of crystalline eltrombopag ethylamine salt.

### DETAIL DESCRIPTION OF INVENTION

The term “about” when used in the present invention preceding a number and referring to it, is meant to designate any value which lies within the range of ±10%, preferably within a range of ±5%, more preferably within a range of ±2%, still more preferably within a range of ±1% of its value. For example “about 10” should be construed as meaning within the range of 9 to 11, preferably within the range of 9.5 to 10.5, more preferably within the range of 9.8 to 10.2, and still more preferably within the range of 9.9 to 10.1.

Room temperature as used herein refers to the temperatures of the thing close to or same as that of the space, e.g., the room or fume hood, in which the thing is located. Typically, room temperature is from about 20° C. to about 30° C., or about 22° C. to about 27° C., or about 25° C. A process or step may be referred to herein as being carried out “overnight”. This refers to a time interval, e.g., for the process or step, that spans the time during the night, when that process or step may not be actively observed. This time interval is from about 8 to about 18 hours, typically about 16 hours.

Unless indicated, the solid state forms of the present invention may be dried. Drying may be carried out, for example, at elevated temperature with or without reduced pressure.

As used herein, the term “vacuum” refers to a reduced pressure of below about 100 mmHg, more preferably below about 50 mmHg, and most preferably below about 30 mmHg.

As used herein, the term “reduced pressure” refers to a pressure below 760 mmHg or 1 atmosphere.

Drying may be suitably carried out in a tray dryer, vacuum oven, Buchi® Rotavapor®, air oven, fluidized bed dryer, spin flash dryer, flash dryer, cone dryer, agitated nutsche filter cum dryer, nauta dryer or the like or any other suitable dryer.

The drying may be carried out at temperature of less than about 150° C., or less than about 120° C., or less than about 100° C., or less than about 70° C., or less than about 60° C., or less than about 50° C., or less than about 40° C., or less than about 20° C., or less than about 0° C., or less than about −20° C. or any other suitable temperature. The drying may be carried out under reduced pressure, that is, less than standard atmospheric pressure or at atmospheric pressure or any other suitable pressure. The drying may take place over a period of about 30 minutes to about 12 hours, or about 2 hours to about 4 hours, or any other suitable time period.

The dried product may be optionally subjected to techniques such as sieving to get rid of lumps before or after drying. The dried product may be optionally milled to get desired particle sizes. Milling or micronization may be performed before drying, or after the completion of drying of the product. Techniques that may be used for particle size reduction include, without limitation, ball, roller and hammer mills, and jet mills.

In some embodiments, the constituent particles may have a particle size distribution such that the constituent particles have a d₁₀ value between about 20 μm and about 100 μm and/or a d₅₀ between 50 μm and 200 μm and/or a d₉₀ value that is between about 150 μm and about 450 μm.

As used herein, the term “d₁₀” refers to the particle size within a distribution of particles where 10 vol. % of the particles have a smaller particle size.

As used herein, the term “d₅₀” refers to the particle size within a distribution of particles where 50 vol. % of the particles have a particle size that is larger and where 50 vol. % of the particles have a particle size that is smaller.

As used herein, the term “d₉₀” refers to the particle size within a distribution of particles where 90 vol. % of the particles have a smaller particle size.

Unless specified otherwise, the word “pure” as used herein means that the material is at least about 99% pure. In general, this refers to purity with regard to unwanted residual solvents, reaction by-products, impurities, and unreacted starting materials. “Substantially pure” as used herein means at least about 98% pure and, likewise, “essentially pure” as used herein means at least about 95% pure.

“Substantially free of one or more of its corresponding impurities” as used herein, unless otherwise defined refers to the compound that contains at least less than about 2%, or less than about 1%, or less than about 0.5%, or less than about 0.3%, or less than about 0.2%, or less than about 0.1%, or less than about 0.05%, or less than about 0.03%, or less than about 0.01%, by weight, of each individual.

Crystalline intermediates are advantageous because impurities are typically removed or substantially reduced during the crystallization process thereby resulting in an intermediate having improved purity. Moreover, crystalline materials often have improved storage stability and better handling properties. Accordingly, it would be desirable to provide an intermediate, useful for preparing eltrombopag, which is in crystalline form.

Accordingly, the present invention provides a process for preparation of eltrombopag olamine (II),

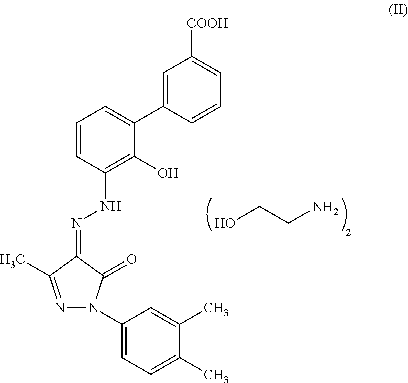

which comprises,
a) diazotizing 3′-Amino-2′-hydroxy-biphenyl-3-carboxylic acid (III) and then condensation with 2-(3,4-Dimethyl-phenyl)-5-methyl-1,2-dihydro-pyrazol-3-one (IV) to give eltrombopag (I);

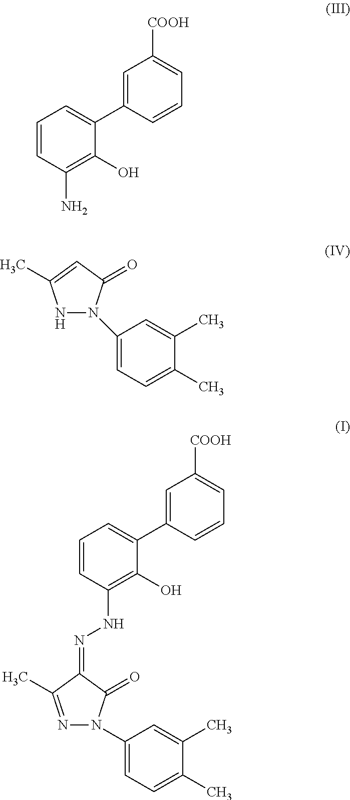

b) converting eltrombopag (I) to eltrombopag salt (V); and

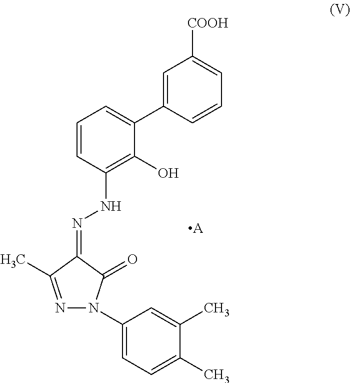

wherein A is base molecule;
c) converting eltrombopag salt (V) to eltrombopag olamine (II).

Base molecule is selected from triethylamine, diisopropyl ethylamine, ethylamine, methylamine, diethylamine and the like. Eltrombopag salt (V) prepared are preferably N-alkyl amine salts, such as, but not limited to, triethylamine, diisopropyl ethylamine, ethylamine, methylamine, diethylamine and the like salts. The term ‘alkyl’ refers to straight, branched chain or cyclic hydrocarbyl groups having from 1 to about 20 carbon atoms, preferably 1 to about 10 carbon atoms.

In step a), diazotization of 3′-Amino-2′-hydroxy-biphenyl-3-carboxylic acid (II) may be carried out by reacting 3′-Amino-2′-hydroxy-biphenyl-3-carboxylic acid in presence of nitrite with acid in suitable solvent. The reaction may be carried out in the presence of alkali metal nitrite such as sodium nitrite, potassium nitrite and the like; alkaline earth metal nitrite such as calcium nitrite and the like; alkyl nitrite such as amyl nitrite, isoamyl nitrite, butyl nitrite, isobutyl nitrite and the like. Preferably, sodium nitrite is used. The acid may be selected from the group consisting of hydrochloric acid, sulfuric acid, hydrobromic acid, acetic acid, silica sulfuric acid and the like. Preferably, hydrochloric acid is used. Solvent is selected from water; ether such as methyl tertbutyl ether (MTBE); nitrile such as acetonitrile; aromatic hydrocarbon such as toluene; ether such as tetrahydrofuran (THF); ketone such as acetone, methyl isobutyl ketone (MIBK), methyl ethyl ketone (MEK); alcoholic solvent C₁₋₄ alcohol such as methanol, ethanol, isopropanol, propanol, butanol and the like; or mixtures thereof. Preferably alcohol such as methanol is used for this reaction. The reaction may be carried out at a temperature in the range of about 0° C. to about 10° C. The reaction is carried out for a period of about 15 minutes to about 8 hours.

Condensation of diazotized product of compound III and with 2-(3,4-Dimethyl-phenyl)-5-methyl-1,2-dihydro-pyrazol-3-one (IV) may be carried out in presence of base. Base may be added before or along with addition of compound IV. Base may be inorganic or organic. Inorganic base may be selected from hydroxide of alkali or alkaline earth metal such as sodium hydroxide, potassium hydroxide, lithium hydroxide, and like; carbonate of alkali or alkaline earth metal such as cesium carbonate, potassium carbonate, sodium carbonate, and like; bicarbonate of alkali or alkaline earth metal such as sodium bicarbonate, potassium bicarbonate and the like; or mixture thereof. Organic base may be selected from triethylamine (TEA), diethylamine (DEA), pyridine, ethanolamine, diisopropylethylamine, methylamine, ethylamine, sodium acetate, potassium acetate, and the like, or mixtures thereof. The reaction may be carried out at a temperature in the range of about 25° C. to about the reflux temperature of the solvent. The reaction is carried out for a period of about 1 hour to about 10 hours.

In step b), eltrombopag (I) is converted to eltrombopag salt (V) in presence of base. Base used here should be capable of forming salt with eltrombopag. The base may be selected from N-alkyl amine such as triethylamine, diisopropylethylamine, ethylamine, methylamine, diethylamine and the like. Preferably, the salt is eltrombopag triethylamine. The base may be taken in 1 mole equivalent or in excess. The reaction is carried out in presence of organic solvent at ambient temperature 25° C. to 35° C. for about 3 to 4 h. Eltrombopag salt (V) is optionally isolated and purified to remove undesirable impurities, which otherwise remains in the final product, if proceed without the formation of eltrombopag salt (V). The obtained eltrombopag salt (V) may be further recrystallized in order to obtain higher purity. The recrystallization may be performed using procedures generally known in the art. In some embodiments, the salt form may be isolated, for example, by concentrating the reaction mixture, or alternatively, by cooling the reaction mixture (with or without concentrating the mixture first) and isolating the resulting precipitate by filtration. As used herein, the term “isolated” does not require absolute purity, but rather is intended as a relative term. Thus, for example, an isolated compound may be one in which the subject compound is at a higher concentration than in the environment from which it was removed. Salt formation may be performed in presence of suitable solvent. The suitable solvent includes, but not limited to, methyl tertbutyl ether (MTBE), acetonitrile, toluene, chloroform, xylene, chlorobenzene, dimethoxyethane, dichloromethane, dichloroethane, tetrahydrofuran (THF), methyl acetate, ethylacetate, n-propyl acetate, isopropyl acetate, tert-butyl acetate, acetone, methyl isobutyl ketone (MIBK), methyl ethyl ketone (MEK), C₁₋₄ alcohol such as methanol, ethanol, isopropanol, propanol, butanol and the like. The reaction may be carried out at a temperature in the range of about 10° C. to about 40° C. The reaction is carried out for a period of about 1 hour to about 20 hours. Preferably the reaction is carried out at a temperature of about 20° C. to about 35° C. for a period of about 1 hour to about 5 hours. The progress of reaction is monitored on thin layer chromatography (TLC) or by high pressure liquid chromatography (HPLC). Generally it is monitored on TLC.

In step c), eltrombopag salt (V) is reacted with ethanolamine in solvent to give eltrombopag olamine (II). Solvent used for this step is selected from methyl tert-butyl ether (MTBE), acetonitrile, toluene, tetrahydrofuran (THF), ethylacetate, isopropyl acetate, acetone, methyl isobutyl ketone (MIBK), methyl ethyl ketone (MEK), C₁₋₄ alcohol such as methanol, ethanol, isopropanol, propanol, butanol and the like, or mixtures thereof. The process comprises providing a reaction mixture of eltrombopag salt and ethanolamine in the solvent and precipitating eltrombopag olamine (II). The reaction mixture may also be provided by combining eltrombopag salt or a suspension of eltrombopag salt in the solvent and ethanolamine or a solution of ethanolamine in the solvent, wherein the solvent of eltrombopag and ethanolamine may be same or different. After the reaction mixture is provided, it may be further maintained, for example at the same temperature of the combination step or at reflux temperature of solvent used, over a period of about 30 minutes to about 10 hours. Precipitation may be achieved, for example, by cooling the reaction mixture to obtain a suspension comprising eltrombopag olamine (II). The recovery may comprise, for example, filtering the obtained solid from the suspension, washing and drying. Washing may be done with the solvent used in the suspension of eltrombopag or the solution of ethanolamine.

In one preferred embodiment, the present invention provides a process for the preparation of eltrombopag olamine (II), the process comprising: (a) treating a reaction mixture containing eltrombopag (I) with a base to give eltrombopag salt (V); and (b) reacting the eltrombopag salt with ethanolamine to give eltrombopag olamine directly, wherein eltrombopag (I) is not isolated and in-situ converted to eltrombopag salt (V).

The process of present invention is given in scheme 1

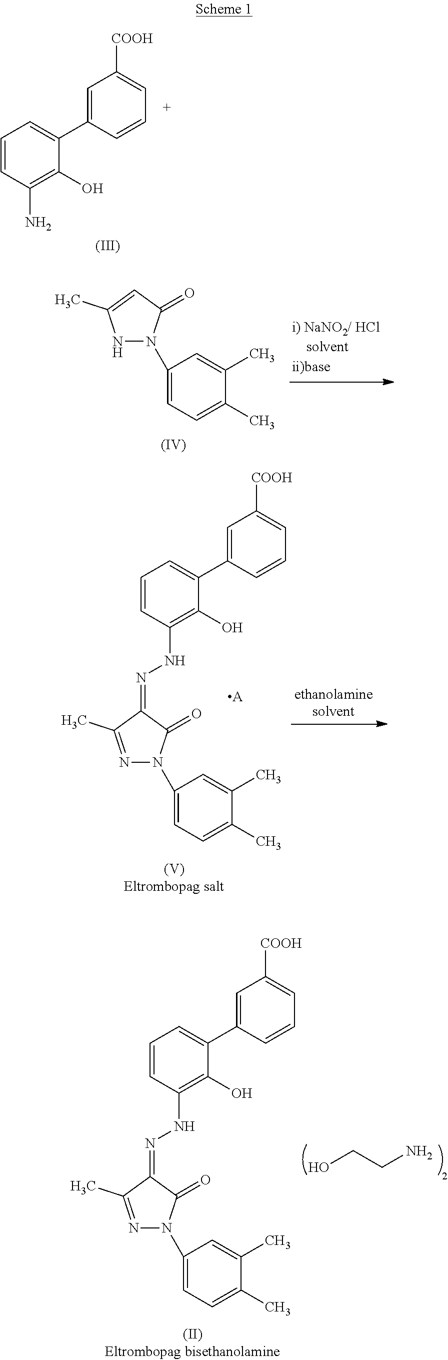

In another embodiment, the present invention provides a process for preparation of eltrombopag salt (V) comprising a step of reacting eltrombopag (I) with a base. Eltrombopag (I) used herein may be direct use of a reaction mixture containing eltrombopag (I) that is obtained in the course of its synthesis; or dissolving eltrombopag (I) in one or more organic solvents. Base used here should be capable of forming salt with eltrombopag. The base may be selected from N-alkyl amine such as triethylamine, diisopropylethylamine, ethylamine, methylamine, diethylamine and the like. Preferably, the salt is eltrombopag triethylamine. The base may be taken in 1 mole equivalent or in excess. Eltrombopag salt (V) is optionally isolated and purified to remove undesirable impurities, which otherwise remains in the final product, if proceed without the formation of eltrombopag salt (V). The obtained eltrombopag salt (V) may be further recrystallized in order to obtain higher purity. The recrystallization may be performed using procedures generally known in the art. In some embodiments, the salt form may be isolated, for example, by concentrating the reaction mixture, or alternatively, by cooling the reaction mixture (with or without concentrating the mixture first) and isolating the resulting precipitate by filtration. As used herein, the term “isolated” does not require absolute purity, but rather is intended as a relative term. Thus, for example, an isolated compound may be one in which the subject compound is at a higher concentration than in the environment from which it was removed. Salt formation may be performed in presence of suitable solvent. The suitable solvent includes, but not limited to, methyl tertbutyl ether (MTBE), acetonitrile, toluene, chloroform, xylene, chlorobenzene, dimethoxyethane, dichloromethane, dichloroethane, tetrahydrofuran (THF), methyl acetate, ethylacetate, n-propyl acetate, isopropyl acetate, tert-butyl acetate, acetone, methyl isobutyl ketone (MIBK), methyl ethyl ketone (MEK), C1-4 alcohol such as methanol, ethanol, isopropanol, propanol, butanol and the like. The reaction may be carried out at a temperature in the range of about 10° C. to about 40° C. The reaction is carried out for a period of about 1 hour to about 20 hours. Preferably the reaction is carried out at a temperature of about 20° C. to about 35° C. for a period of about 1 hour to about 5 hours. The progress of reaction is monitored on thin layer chromatography (TLC) or by high pressure liquid chromatography (HPLC). Generally it is monitored on TLC.

The process of present invention is given in scheme 2:

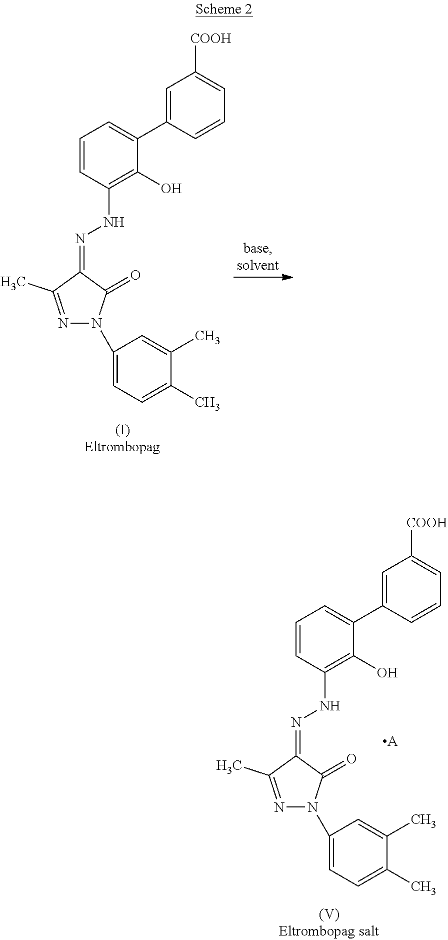

Further, the present invention discloses polymorphic form of eltrombopag salts.

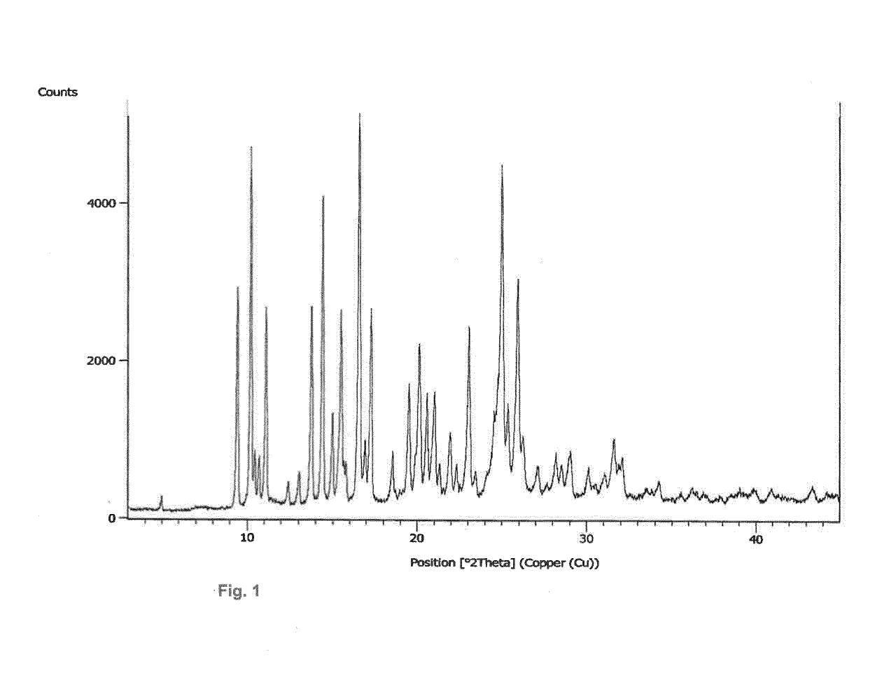

In one embodiment, the crystalline form of eltrombopag triethylamine is characterized by a powder x-ray diffraction pattern comprising diffraction peaks at 2θ values of 4.9, 9.4, 10.2, 10.4, 10.7, 11.1, 12.4, 13.0, 13.7, 14.4, 15.0, 15.5, 16.6, 16.9, 17.3, 18.5, 19.5, 20.1, 20.5, 21.0, 21.3, 21.9, 22.3, 23.0, 23.4, 25.0, 25.3, 25.9, 26.2, 27.1, 27.6, 28.2, 28.5, 29.0, 30.1, 31.0, 31.6, 32.0±0.2° 2θ. In another embodiment, the crystalline form is further characterized by a powder x-ray diffraction pattern substantially in accordance with FIG. 1.

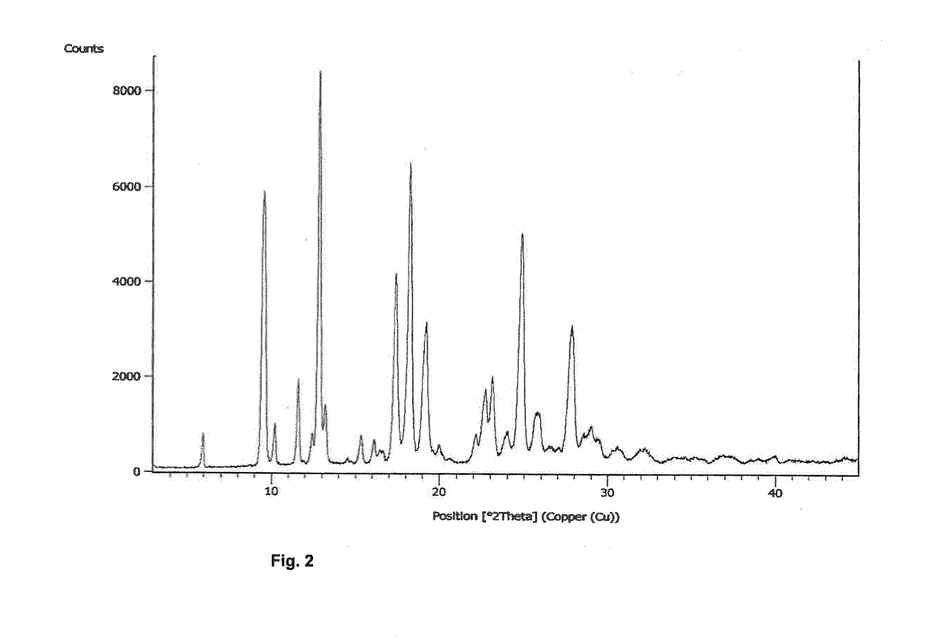

In another embodiment the present invention encompasses crystalline eltrombopag diisopropylamine characterized by an XRPD pattern having peaks at 5.9, 9.4, 10.2, 11.6, 12.4, 12.8, 13.2, 15.3, 16.1, 17.4, 18.3, 19.0, 19.2, 20.0, 22.1, 22.7, 23.1, 24.0, 24.9, 25.6, 27.7, 27.9, 28.5, 29.0, 29.5, 30.6, 31.8±0.2° 2θ. The XRPD pattern is as given in FIG. 2.

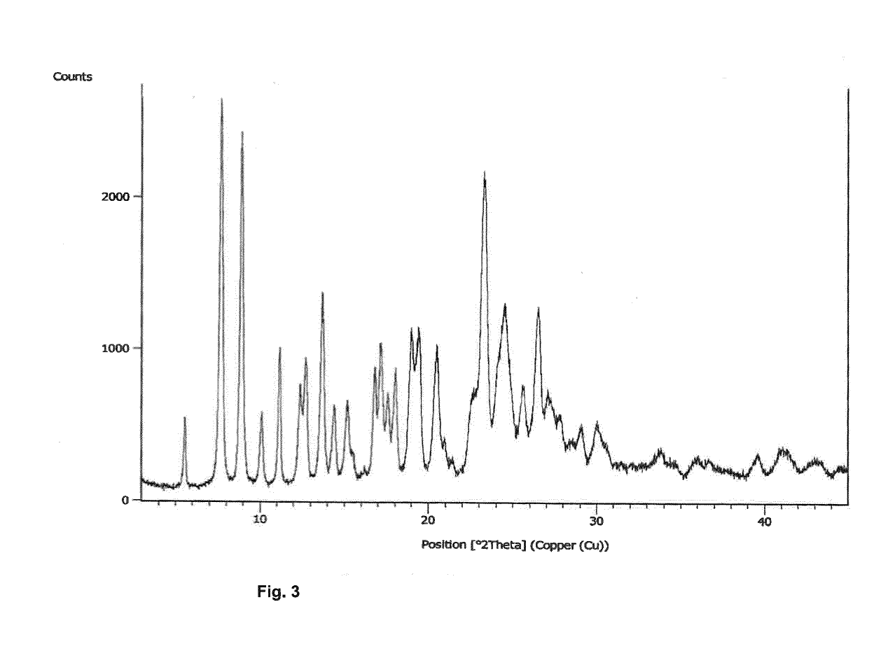

In another embodiment the present invention encompasses crystalline eltrombopag ethylamine characterized by an XRPD pattern having peaks at 5.5, 7.7, 8.9, 10.0, 11.1, 12.3, 12.7, 13.7, 14.4, 15.2, 15.5, 16.8, 17.1, 17.5, 18.0, 18.9, 19.4, 20.4, 20.9, 21.4, 22.5, 23.3, 24.5, 25.6, 26.5, 27.0, 27.8, 29.1, 29.9, 33.8, 36.0, 36.7, 39.5, 40.9±0.2° 2θ. The XRPD pattern is as given in FIG. 3.

Powder X-ray Diffraction can be performed using PANALYTICAL ExpertPro DY666, the powder X-ray diffraction pattern was measured at room temperature using a Cu Kα filled tube (45 kV, 40 mA) as the X-ray source with a wide-angle goniometer, a ½° scattering slit, an programmable divergence slit, and a x'celerator detector. Data collection was done in 2θ continuous scan mode at a scan speed of 0.047747/s in scan steps of 0.0083556° in the range of 3° to 45°.

The following examples are given for the purpose of illustrating the present invention and should not be considered as limitation on the scope or spirit of the invention.

### Example 1

### Preparation of Eltrombopag Triethylamine Salt

To a cooled at 0 to 10° C. mixture of 3′-Amino-2′-hydroxy-biphenyl-3-carboxylic acid (100 g) in methanol (2000 ml) was added aq. hydrochloric acid solution (150 ml conc. hydrochloric acid in 150 ml water). Aq. sodium nitrite solution (31.87 g sodium nitrite in 100 ml water) was added to the reaction mixture at 0° C. to 10° C. within 15 min and stirred further for 1 h. Triethylamine (200 ml) was added to the reaction mixture at 0° C. to 10° C. till pH 6 to 8 was obtained. 2-(3,4-dimethyl-phenyl)-5-methyl-1,2-dihydro-pyrazol-3-one (88.24 g) was added to the reaction mixture and stirred for 2 h at 25° C. to 35° C. After completion of the reaction on TLC, triethylamine (176.57 g) was added to the reaction mixture and heated at 50° C. to 60° C. for 2 h. The reaction mixture is distilled out under vacuum to get residue. Methanol (1000 ml) was added to the residue and heated at 50° C. to 60° C. for 30 min. The reaction mixture was cooled to 25° C. to 35° C. for 2 h. The suspension was filtered and obtained solid was washed with methanol (2×50 ml) to give wet cake. Water (1000 ml) was added to this wet cake and heated at 50° C. to 60° C. for 1 h. The reaction mixture is filtered, obtained solid was washed with water (2×50 ml) and suck dried to give wet cake. Water (1000 ml) was added to this wet cake and heated at 50° C. to 60° C. for 1 h. The reaction mixture was filtered, obtained solid was washed with water (2×50 ml) and suck dried. The solid was dried at 60° C. to 70° C. for 12-15 h to give eltrombopag triethylamine salt (180.0 g)

Yield: 76.0%

Purity by HPLC: 99.6%

¹H NMR (DMSO) δ: 1.08-1.11 (9H, t), 2.19-2.33 (6H, m), 2.38 (3H, s), 2.87-2.92 (6H, q), 7.06-7.13 (3H, t), 7.55-7.65 (2H, d), 7.74-7.89 (4H, d), 8.16-8.23 (1H, d)

Triethylamine content by titration: 18.5%

The XRPD of above obtained Eltrombopag triethylamine salt is given in FIG. 1.

### Example 2

### Preparation of Eltrombopag Bisethanolamine Salt

To a stirred solution of ethanol amine (22.47 g) in methanol (1000 ml) was added eltrombopag triethylamine salt (100 g) lot wise within 30 min at 25° C. to 35° C. and stirred further for 5 h. The reaction mixture was heated to 50° C. to 60° C. and stirred for 1 h. The reaction mixture was cooled to 25° C. to 35° C. and stirred for 2 h. The reaction mixture was filtered, obtained solid was washed with methanol (5 ml×2) and then suck dried to give wet cake. The solid is dried in air tray dryer for 4-5 h to give eltrombopag bisethanolamine salt (90.0 g).

Yield: 87.0%

Purity by HPLC: 99.8%

¹H NMR (DMSO) δ: 2.26 (3H, s), 2.30 (3H, s), 2.37 (3H, s), 2.89-2.92 (4H, t), 3.66-3.68 (4H, t), 6.97-7.01 (1H, t), 7.13-7.16 (2H, m), 7.42-7.46 (1H, t), 7.55-7.57 (1H, dd), 7.63-7.69 (3H, m), 7.90-7.93 (1H, m), 8.09-8.10 (1H, t)

Bisethanolamine content by titration: 21.5%

### Example 3

### Preparation of Eltrombopag Bisethanolamine Salt

To a stirred ethanol amine (300 ml) was added eltrombopag triethylamine salt (100 g) lot wise within 30 min at 25° C. to 35° C. The solution was stirred further for 1 h at RT. The reaction mixture was heated to 50° C. to 60° C. and methanol (500 ml) was added drop wise within 30 min to get precipitates. The reaction mixture was cooled to 25° C. to 35° C. and stirred for 3 h. The obtained suspension was filtered, the solid obtained was washed with methanol (5 ml×2) and then suck dried to give wet cake. The solid was dried in air tray dryer at 50° C. for 4-5 h to give eltrombopag bisethanolamine salt (85.0 g).

Yield: 81.73%

Purity by HPLC: 99.8%

Ethanolamine content by titration: 21.7%

### Example 4

### Preparation of Eltrombopag Triethylamine Salt

Eltrombopag (10.0 g) was added to tetrahydrofuran (THF) (1000 ml) under stirring at 25° C. to 35° C. to get a dark red color solution. Triethylamine (5.7 g) was added slowly to above prepared reaction mixture and stirred for 3 hours at 25° C. to 35° C. The reaction mixture was filtered and obtained solid was washed with THF (5 ml×2) and suck dried. The solid was dried in air oven at 50° C. to 60° C. for 12 h to get the title product (9.7 g)

Yield: 80.0%

Purity by HPLC: 99.5%

Triethylamine content by titration: 18.5%

### Example 5

### Preparation of Eltrombopag Diisopropylethylamine Salt

Eltrombopag (10.0 g) was added to tetrahydrofuran (THF) (1000 ml) under stirring at 25° C. to 35° C. to get a dark red color solution. Diisopropylethylamine (7.30 g) was added slowly to above prepared reaction mixture and stirred at 25° C. to 35° C. for 3 h. The reaction mixture was filtered and obtained solid was washed with THF (5 ml×2) and suck dried. The solid was dried in air oven at 50° C. to 60° C. for 12 h to get the title product (9.8 g)

Yield: 76.0%

¹H NMR (DMSO) δ: 1.13-1.15 (15H, d), 2.22-2.52 (11H, m), 2.88 (2H, s), 7.06-7.13 (3H, t), 7.56-7.89 (6H, m), 8.15-8.23 (1H, d)

Diisopropylethylamine content by titration: 21.40%

The XRPD of above obtained eltrombopag diisopropylamine salt is given in FIG. 2.

### Example 6

### Preparation of Eltrombopag Ethylamine Salt

Eltrombopag (10.0 g) was added to tetrahydrofuran (THF) (1000 ml) under stirring at 25° C. to 35° C. to get a dark red color solution. Ethyl amine (2.55 g) was added slowly to above prepared reaction mixture and stirred for 3 hours at 25° C. to 35° C. The reaction mixture was filtered and obtained solid was washed with THF (5 ml×2) and suck dried. The solid was dried in air oven at 50° C. to 60° C. for 12 h to get the title product (8.5 g)

Yield: 77.0%

The XRPD of above obtained eltrombopag ethylamine salt is given in FIG. 3.

### Example 7

### Preparation of Eltrombopag Bisethanolamine Salt

Eltrombopag bisethanolamine salt can be prepared from eltrombopag diisopropylethylamine salt or Eltrombopag ethylamine salt or from any such other N-alkylamine salts by following the procedure of example 2.

### Example 8

### Preparation of Eltrombopag Triethylamine Salt

To a cooled at 0 to 10° C. mixture of 3′-Amino-2′-hydroxy-biphenyl-3-carboxylic acid (100 g) in methanol (2000 ml) was added Aq. sodium nitrite solution (31.9 g sodium nitrite in 100 ml water) then aq. hydrochloric acid solution (142 gm conc. hydrochloric acid in 150 ml water) was added to the reaction mixture at 0° C. to 10° C. within 15 min and stirred further for 1 h. Triethylamine (264.8 gm) was added to the reaction mixture at 0° C. to 10° C. till pH more than 10 was obtained. 2-(3,4-Dimethyl-phenyl)-5-methyl-1,2-dihydro-pyrazol-3-one (88.2 g) was added to the reaction mixture and stirred for 2 h at 25° C. to 35° C. Reaction was monitored by TLC for the completion. The reaction mixture was further heated at 50° C. to 60° C. and stirred for 2 h. The reaction mixture was dried under vacuum to obtain residue. Methanol (1000 ml) was charged and the mixture was stirred for 2 h. Solid was filtered and washed with methanol (2×50 ml). The wet cake was further stirred with water (1000 ml) for 1 h, filtered and washed with water (2×50 ml). The solid was dried in air oven at 50° C. to 60° C. for 12 h to get the title product (180 g).

1. A process for preparation of eltrombopag olamine (II), 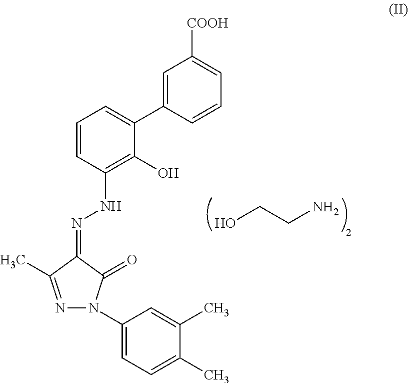
comprising steps of:
a) Diazotizing 3′-Amino-2′-hydroxy-biphenyl-3-carboxylic acid (III) and then condensation with 2-(3,4-Dimethyl-phenyl)-5-methyl-1,2-dihydro-pyrazol-3-one (IV) to give eltrombopag (I); 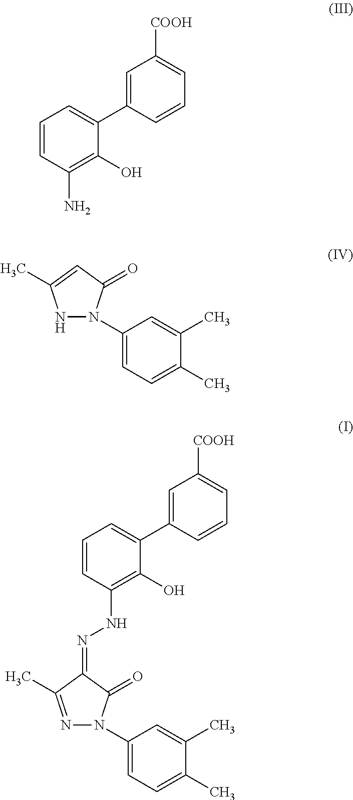
b) converting eltrombopag to eltrombopag salt (V) 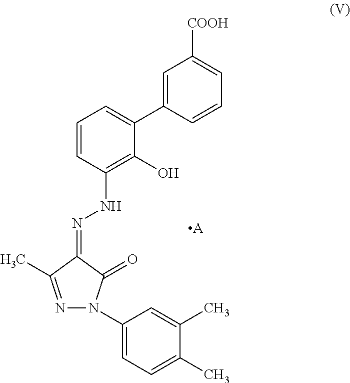
wherein A is base molecule; and
c) converting eltrombopag salt (V) to eltrombopag olamine (II).

2. The process according to claim 1, wherein step a) is carried out in presence of nitrite, acid and solvent.

3. The process according to claim 2, wherein nitrite is selected from sodium nitrite, potassium nitrite, calcium nitrite, amyl nitrite, isoamyl nitrite, butyl nitrite, isobutyl nitrite; acid is selected from hydrochloric acid, sulfuric acid, hydrobromic acid, acetic acid, silica sulfuric acid; and solvent is selected from water; ether such as methyl tertbutyl ether (MTBE); nitrile such as acetonitrile; aromatic hydrocarbon such as toluene; ether such as tetrahydrofuran (THF); ketone such as acetone, methyl isobutyl ketone (MIBK), methyl ethyl ketone (MEK); alcoholic solvent C₁₋₄ alcohol such as methanol, ethanol, isopropanol, propanol, butanol or mixtures thereof.

4. The process according to claim 1, wherein step a) is carried out in presence of base selected from inorganic base hydroxide of alkali or alkaline earth metal such as sodium hydroxide, potassium hydroxide, lithium hydroxide; carbonate of alkali or alkaline earth metal such as cesium carbonate, potassium carbonate, sodium carbonate; bicarbonate of alkali or alkaline earth metal such as sodium bicarbonate, potassium bicarbonate or mixtures thereof; and organic base selected from triethylamine (TEA), diethylamine (DEA), pyridine, ethanolamine, diisopropylethylamine, methylamine, ethylamine, sodium acetate, potassium acetate or mixtures thereof.

5. The process according to claim 1, wherein step b) is carried out in presence of base selected from N-alkyl amine such as triethylamine, diisopropylamine, diisopropyl ethylamine, ethylamine, methylamine, and diethylamine.

6. The process according to claim 1, wherein step c) is carried out in presence of solvent selected from methyl tert-butyl ether (MTBE); acetonitrile; toluene; tetrahydrofuran (THF); ethylacetate; isopropyl acetate; acetone; methyl isobutyl ketone (MIBK); methyl ethyl ketone (MEK); C₁₋₄ alcohol such as methanol, ethanol, isopropanol, propanol, butanol; or mixtures thereof.

7. The process according to claim 1, wherein step b), eltrombopag (I) is not isolated and in-situ converted to eltrombopag salt (V).

8. A salt of eltrombopag having structural formula (V): 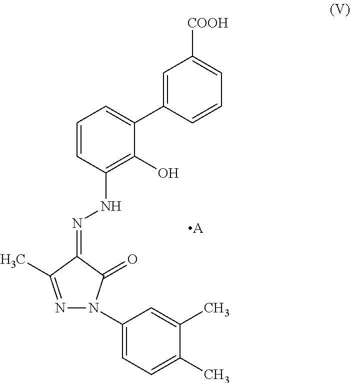
wherein A is N-alkyl amine selected from triethyamine, diisopropylamine, diisopropyl ethylamine, ethylamine, and methylamine.

9. (canceled)

10. A crystalline eltrombopag triethylamine characterized by a powder x-ray diffraction pattern comprising diffraction peaks at 20 values of 4.9, 9.4, 10.2, 10.4, 10.7, 11.1, 12.4, 13.0, 13.7, 14.4, 15.0, 15.5, 16.6, 16.9, 17.3, 18.5, 19.5, 20.1, 20.5, 21.0, 21.3, 21.9, 22.3, 23.0, 23.4, 25.0, 25.3, 25.9, 26.2, 27.1, 27.6, 28.2, 28.5, 29.0, 30.1, 31.0, 31.6, 32.0±0.2° 2θ.

11.-15. (canceled)
